(19) United States
(12) Patent Application Publication
(10) Pub. No.: US 2024/0149011 A1
(43) Pub. Date: May 9, 2024

(54) OPTIMIZING SLEEP PERSONALIZATION USING LEARNING METHODS ACROSS MULTIPLE USERS AND BED PLATFORMS

(71) Applicants: Ely Tsern, Los Altos, CA (US); Jonathon Farringdon, Pitssburg, PA (US)

(72) Inventors: Ely Tsern, Los Altos, CA (US); Jonathon Farringdon, Pitssburg, PA (US)

(21) Appl. No.: 18/495,698

(22) Filed: Oct. 26, 2023

Related U.S. Application Data
(60) Provisional application No. 63/382,772, filed on Nov. 8, 2022.

(51) Int. Cl. A61M 21/02; A47C 31/12; G16H 40/67

(52) U.S. Cl. CPC A61M 21/02; A47C 31/123; G16H 40/67; A61M 2021/0022

(57) ABSTRACT

One or more aspects of a sleep environment of a sleep platform for a user may be set or modified based on at least some sleep environment information for sleep platforms of other users. The other users may have similar characteristics as the user. The aspects of the sleep environment may be set or modified based on one or more aspects of sleep environments of sleep platforms of the other users indicated as providing improved sleep quality. The sleep environment of the sleep platform may include sleep surface conditions and/or conditions surrounding the sleep surface.

### CROSS REFERENCE TO RELATED APPLICATION

This application claims the benefit of the filing date of U.S. Provisional Patent Application No. 63/382,772, filed on Nov. 8, 2022, the disclosure of which is incorporated by reference herein.

### FIELD OF THE INVENTION

The present invention relates generally to sleep environments, and more particularly to optimization of a sleep environment based on information of sleep environments for a plurality a sleep environments and outcome metrics for a plurality of users using those sleep environments.

### BACKGROUND OF THE INVENTION

Sleep is a universal need for people. Sleep provides many physiological benefits, and a sound night's sleep is often desired by many. Unfortunately, some may not obtain good quality sleep, even when sufficient time and preparation for sleep is available.

A sleep environment of the sleep platform may affect quality of sleep. For example, firmness of a sleep surface of the sleep platform, temperature of the sleep surface, sound and lighting in an area of the sleep platform, and other aspects of the sleep environment may all impact the quality of sleep. Moreover, during a night's sleep, variation in these aspects of the sleep environment may affect quality of sleep, positively or negatively. Appropriate control of a sleep environment to increase quality of sleep may therefore be difficult to determine. In addition, there may be differences and difficulties in defining and measuring sleep quality, which may further hinder determining appropriate control of a sleep environment.

### BRIEF SUMMARY OF THE INVENTION

In some embodiments one or more aspects of a sleep environment of a sleep platform for a user is set or modified based on at least some sleep environment information for sleep platforms of other users, the user and the other users having similar characteristics, with the aspects of the sleep environment set or modified based on one or more aspects of sleep environments of sleep platforms of other users indicated as providing improved sleep quality.

In some embodiments the sleep environment of the sleep platform for the user is adjusted based on sleep environment information for those of the other users whose sleep quality exceeds that of the user. In some embodiments the sleep quality is determined by one or more measurements that may be made of the user and the other users during sleep sessions of those users. In some embodiments the sleep quality is in addition or instead determined based on perceptions of sleep quality by those users or subjective evaluation of the user's restorative sleep quality. In some embodiments, a metric of restorative sleep quality is used to determine the user's perceived sleep quality. In some embodiments restorative sleep quality is measured with a user questionnaire based on a series of questions that evaluate the user's state of restoration, refreshed feeling, energy, mental acuity, and/or emotional state. In some embodiments the questions are questions that have been validated as relevant to measuring restorative sleep quality, for example by way of an academic study.

In some embodiments at least one server receives information regarding status of the sleep platforms, the at least one server receives metrics indicating quality of sleep for the plurality of users of sleep platforms, the at least one server determines modifications to the status of at least one of the sleep platforms by selecting settings for the at least one of the sleep platforms based on settings for sleep platforms of users with a sleep quality of at least a predetermined level.

In some embodiments sleep related data is collected for a plurality of users, sleep profile settings indicative of higher sleep quality are determined, and sleep environment for a sleep platform is modified using the sleep profile settings indicative of higher sleep quality. In some embodiments sleep profile settings indicative of higher sleep quality are propagated to one or more sleep platforms for use in testing whether those profile settings result in higher quality sleep for users of the one or more sleep platforms.

These and other aspects of the invention are more fully comprehended upon review of this disclosure.

### BRIEF DESCRIPTION OF THE FIGURES

FIG. 1 is a semi-block diagram of a bed in a network, in accordance with aspects of the invention.

FIG. 2 is a semi-block diagram of a system including a plurality of sleep platforms coupled with a server by a network, in accordance with aspects of the invention.

FIG. 3 is a flow chart of a process for determining settings for conditioning an environment of a sleep platform based on user characteristics, in accordance with aspects of the invention.

FIG. 4 is a flow chart of a process for updating a sleep profile for a user based on sleep profile information of other users with high sleep quality, in accordance with aspects of the invention.

FIG. 5 is a flow diagram of a process for determining test sleep profile parameters based on sleep quality results from a plurality of users, in accordance with aspects of the invention.

FIG. 6 is a flow diagram of a process for testing sleep profile changes for a user, in accordance with aspects of the invention.

### DETAILED DESCRIPTION

A sleep platform may include and/or be associated with various components for adjusting or controlling an environment of the sleep platform. The environment of the sleep platform may include one, some, or all of sleep surface firmness, sleep surface temperature or temperatures across different regions of the sleep surface or sleeper's body, lighting in an environment of the sleeper and sleep platform, room air temperature and humidity for a room containing the sleep platform, aromas surrounding the sleep platform, and possibly other conditions. The sleep platform may command and/or request adjustments to the environment of the sleep platform based on one or more sleep profiles. The sleep profiles may include information related to a desired environment of the sleep platform during and at times and/or stages of use of the sleep platform by a user, for example for sleeping, and in some embodiments preparing for sleep and/or awakening.

In some embodiments, at least some information of one or more of the sleep profiles for the user is determined based on information of sleep profiles of other users who meet at least one criteria for sleep quality. In some embodiments the criteria is for example a length of time asleep, sleep efficiency, which in some embodiments is a percentage of time the user is asleep during the night, or percentage of time asleep in a particular sleep stage. In some embodiments the sleep quality criteria is for example a subjective perceived quality of sleep of users based on their answers to questions on their sleep and resulting impact on their waking hours, such as how refreshed, energetic, or restored they feel after their sleep. In some embodiments, the sleep quality criteria is the metric of restorative sleep. In some embodiments the metric of restorative sleep is defined as in R. Robbins et al., A Nationally Representative Survey Assessing Restorative Sleep in US Adults, Front. Sleep (21 Jul. 2022). In some embodiments, for example, a sleep profile for may be modified to reflect, or more closely reflect, sleep profiles of other users who have achieved a greater level of sleep quality.

In some embodiments users are grouped by one or more characteristics, and an initial or default sleep profile is provided for a new user based on information of sleep profiles of other users in a same group as the user, with the other users being users who meet at least one criteria for sleep quality. In some embodiments default sleep profiles may be changed over time. For example, in some embodiments an initial default sleep profile may be changed to a new default sleep profile in response to ascertaining that changes to the initial default sleep profile lead to generally improved sleep for users in a group. In some embodiments correlations between higher sleep quality and sleep profile entries of users is determined, and sleep profile entries indicative of higher sleep quality are used as test modifications of sleep profiles for users.

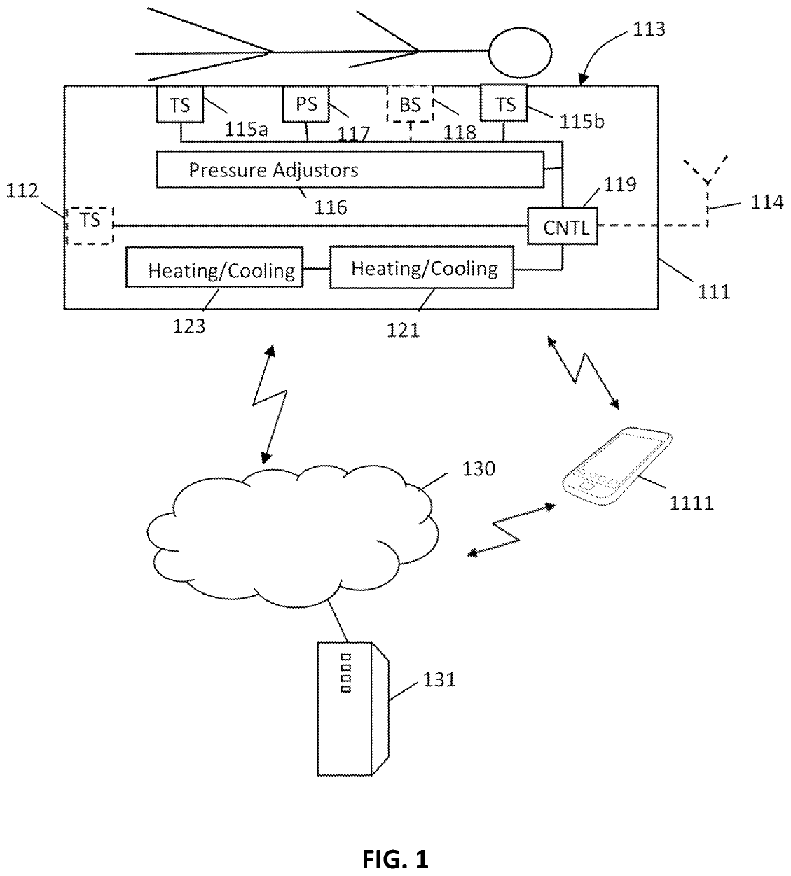

FIG. 1 is a semi-block diagram of a bed 111 in a network, in accordance with aspects of the invention. In some embodiments the bed, including its various components, may be considered a sleep platform. The bed of FIG. 1 includes a sleep surface 113 as an upper surface. In various embodiments, the sleep surface may be a top surface of a mattress, and in some embodiments the mattress, which itself may be comprised of multiple parts (separable or inseparable) may sit on top of a foundation, with the mattress and foundation considered the bed. In various embodiments, however, the bed may include other parts, and in some embodiments the various parts may be combined into one or more separable or non-separable items. The bed of FIG. 1 may be generally rectangular parallelepiped in form, although other forms may instead be used, and in various embodiments may house a variety of components and materials and be comprised of multiple separable components and/or layers. Generally, a user, or multiple users depending on the bed, sleeps on the sleep surface.

The bed of FIG. 1 includes components for conditioning a sleep environment. For the example bed of FIG. 1, the components include a heating/cooling component 121 (and optional heating/cooling component 123) and a pressure adjustment component 116. The heating/cooling component allows for adjustment of temperature of the sleep surface of the bed or in some embodiments an environment under the blankets of the sleeper. The pressure adjustment component allows for adjustment of firmness of the sleep surface or, in some embodiments, across various zones of the sleep surface. In some embodiments the bed may be in communication with local devices, for example one or more aromatherapy devices and/or air filtrations devices (both not shown in FIG. 1) and/or media devices (not shown in FIG. 1) and/or connected mobile devices and/or room lighting control devices and/or room or dwelling temperature control devices. These local devices also may condition the sleep environment, in various embodiments.

In some embodiments the heating/cooling component comprises a thermoelectric device, for example a Peltier device. In some embodiments the heating/cooling device comprises a heat pump. In some embodiments, the heating/cooling component may just be a heating component, for example a resistive heater, which in some embodiments may be adjacent or part of the sleep surface. In some embodiments, the heating/cooling component may just be a cooling component, for example an air conditioning device, which in some embodiments may be adjacent or part of the sleep surface. In some embodiments, the heating and/or cooling system includes combinations of heating and/or cooling devices. In the embodiment of FIG. 1 the heating/cooling component is illustrated within the bed, away from the sleep surface. In such embodiments, passageways allowing for thermal transfer between the heating/cooling component and the sleep surface may be provided. For example, in some embodiments, airway passageways are provided between the heating/cooling component and the sleep surface, and some embodiments include other components, for example one or more fans, to assist in conducting heat towards or away from the sleep surface. In some embodiments, where other fluids, for example liquids or fluids that may change between a gaseous and liquid state during use, may be used to control the temperature of the sleep surface, other fluid pathway structures, such as tubes, may be used to move fluids between the active heating and/or cooling component and the sleep surface. Collectively, in some embodiments the temperature control system can include heating and/or heating/cooling components and other fluid (including gaseous fluids) control components, such as fans. The bed may also include a second heating/cooling component 123. The use of a second heating/cooling component may be desirable, for example, in providing differentiation in temperature between different portions of the sleep surface, for example for different sides of the bed used by different users for sleep in a bed normally used by two people. In other embodiments vents or other devices or structures instead or in addition may be used to vary temperature across the sleep surface.

In some embodiments the pressure adjustment component comprises an array of controllable bladders or coils under the sleep surface of the bed. In some embodiments each of the bladders/coils or groups of bladders/coils are individually adjustable, so as to provide a different level of firmness to the surface of the bed. In some embodiments, the pressures across the surface can be modulated according to different pre-determined patterns to create various motion experiences for the user to help them relax or fall asleep. These motion patterns can also by synchronized to other environment events controlled by the sleep platform, such as media content over audio or video displays on the user's mobile or connected devices in the room, lighting. These patterns or their synchronized events, or in some embodiments specific media content, possibly in combination with the synchronized events, can be personalized and optimized for each user, in some cases optimized for improved sleep quality metrics, and part of the user's sleep profile.

The components for conditioning the sleep environment are generally commanded to do so by a controller 119. In most embodiments the controller utilizes information of one or more sleep profiles to determine configuration of an environment of the sleep platform, for example as conditioned by the components of the bed. In addition, in some embodiments the controller may request or command conditioning of the sleep environment by components independent of or external to the bed.

The controller 119 of the bed of FIG. 1 is coupled to a network 130 by way of wired or wireless communication circuitry, which may include for example antenna 114. In such embodiments the controller may be coupled to a remote server 131 (for example by a network 130, which may include the Internet and, in some embodiments, a local area network). In some embodiments the controller may provide information regarding the sleep profile, bed settings, and, in some embodiments, user sleep information to the server, and the controller may receive sleep profile information from the server. In such embodiments the controller may also be coupled to components external to the bed, for example lighting, audio, and/or aromatherapy devices, directly or by way of the local network. As further illustrated in FIG. 1, the controller may also communicate with a sleeper's personal compute device, for example a smartphone 1111, a tablet computer, or other device, either directly, for example using Bluetooth communications, through a local network device (not shown in FIG. 1), or through the remote server.

The sleep profiles may, for example, specify desired pressures and temperatures for the sleep surface of the bed. In various embodiments the specified pressures and temperatures may be different at different times during a sleep session, or an expected or desired sleep session, and the sleep profiles may specify times for the specified pressures and temperatures. In some embodiments the specified times may be absolute times, for example 1:03 AM local time, but more generally the specified times may be relative to entry time to the bed of the user, after the user begins sleep, or, in various embodiments dependent on a sleep stage of the user.

In generating commands, the controller may do so based on information of the sleep profiles and using information from sensors, for example temperature sensors 115a,b, pressure sensors 117, and, in some embodiments, biometric sensors 118. The temperature sensors may be positioned in or adjacent the sleep surface, and provide an indication of a temperature of the sleep surface. In some embodiments, the temperature sensors are worn by the sleeper, provide an indication of a temperature of the sleeper's body or portion of body where the sensor is worn, and can be wired or wirelessly connected to the controller. The pressure sensors may be located under the sleep surface, and provide an indication of pressures on or across the sleep surface. Alternatively, the pressure sensors may be located in the controller, for example near the control valves and pneumatically connected to air chambers underneath the sleep surface, for instance via air tubes, to measure the pressure in the air chambers. The biometric sensors may be located in or under the sleep surface, and may provide an indication of heart rate, breathing information, movement, or other biometric information regarding the user on the sleep surface. In some embodiments the biometric sensors may be in an article worn by the user, for example a shirt, with the biometric sensors wirelessly communicating with the controller. In some embodiments the biometric sensors are as discussed or part of an item as discussed in J. Kelly et al., Recent Developments in Home Sleep-Monitoring Devices, ISRN Neurology, vol. 2012, article ID 768794, the disclosure of which is incorporated herein for all purposes.

The controller also may make use of additional information in generating commands, for example time-of-day information (for example maintained by the controller), information provided by users by way of user devices, and historical usage and/or sensor information maintained by the controller. In some embodiments the controller may command conditioning of the sleep environment as discussed in U.S. patent application Ser. No. 16/401,108, entitled SLEEP PHASE DEPENDENT TEMPERATURE CONTROL AND LEARNING METHODS TO OPTIMIZE SLEEP QUALITY, filed on May 1, 2019, and/or U.S. patent application Ser. No. 16/401,064 entitled SLEEP PHASE DEPENDENT PRESSURE CONTROL AND LEARNING METHODS TO OPTIMIZE SLEEP QUALITY, filed on May 1, 2019, the disclosures of both of which are incorporated by reference for all purposes herein.

As illustrated in FIG. 1, the controller is housed within the bed. In various embodiments the controller can be housed in either the mattress, base or be located externally outside of the bed. In some embodiments the controller comprises one or more processors. In some embodiments the controller is comprised of more than one processor, and the controller may be partitioned and housed in at least two separate physical enclosures, each with at least one processor. In some embodiments the controller is comprised of more than one processor, and the controller may be partitioned and housed in at least two separate physical enclosures, each with at least one processor. The controller may also include pneumatic valves and pumps that control the air flow to the air coils or bladders that adjust the sleep surface pressures. These pneumatic valves and pumps may be controlled by the one or more processors.

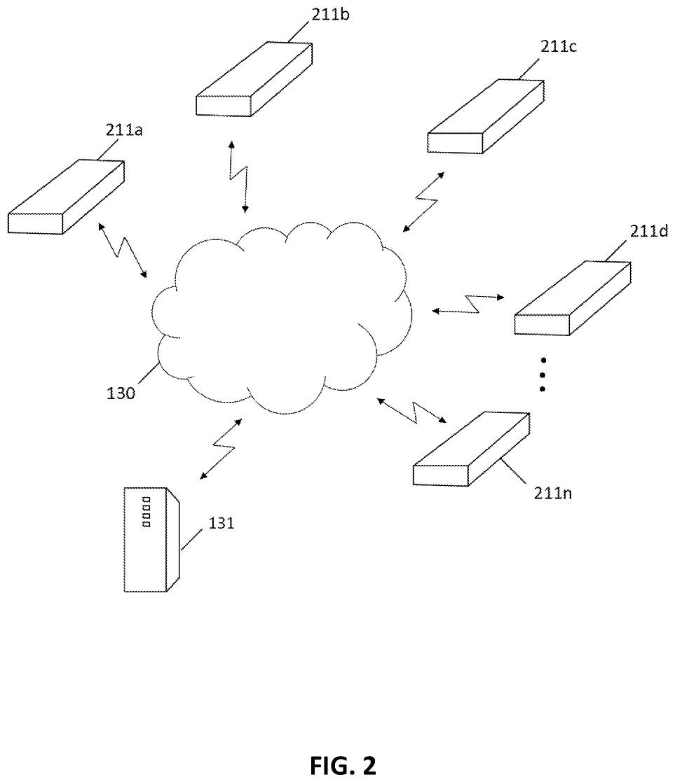

FIG. 2 is a semi-block diagram of a system including a plurality of sleep platforms 211a-n coupled with a server 131 by a network 130. In some embodiments the sleep platforms 211a-n are the bed discussed with respect to FIG. 1. In some embodiments the server is the remote server discussed with respect to FIG. 1.

In some embodiments each of the sleep platforms 211a-n is associated with a corresponding user. In some embodiments a user may use different sleep platforms for different sleep sessions, such that a particular bed may be associated with different users at different times. Moreover, in some embodiments a sleep platform may be associated with more than one user, for example if two users share the same sleep platform for a sleep session.

Each user may have one or more sleep profiles associated with the user, and each of the sleep platforms may condition a sleep environment of the sleep platform in accordance with one or more sleep profiles. The sleep profiles, at least initially, may be based on information provided by the user, for example user physical information and sleep preferences. In some embodiments, user physical information and sleep preferences is obtained by way of questionnaires provided to users, for example by way of forms provided by a server to a computer, tablet, or smartphone of the user, with responses returned to the server. The sleeper profiles may also be based on historical information of the sleeper, either collected on the sleep platform or collected by means of another health and/or sleep tracking or monitoring device or platform. The server may assign users to one of a plurality of groups. In some embodiments the server may assign users to a group based on capabilities of the capabilities of the sleep platform used by the users. In some embodiments users with a sleep platform having the same capabilities, or in some embodiments similar capabilities, may be assigned to a same group. In some embodiments sleep platforms may be considered to have similar capabilities if they commonly have, in various embodiments, one, some, or all of sleep surface pressure control capabilities, sleep surface temperature control capabilities, and/or other sleep environment conditioning capabilities. In addition, or instead, in some embodiments the server may assign users to a group based on physical information of the users and/or sleep preferences of the users. For example, based on the physical information and sleep preferences, the server may assign users to one of a plurality of groups, with each group having similar characteristics and sleep preferences. For example, a first group may be users who are a certain age group, have a body mass index (BMI) below a predetermined level, have sleep apnea, and prefer to rise early and sleep on their side, and a second group may be users who have a resting pulse rate below a predetermined rate, and prefer to sleep late and on their back. In some embodiments a first default sleep profile may be used for the first group, and a second default sleep profile may be used for the second group.

In addition, in various embodiments the server may change default, or then active or used, sleep profiles over time, for example to include sleep profile settings indicative of higher quality sleep. In some embodiments the server determines sleep profile settings indicative of higher quality sleep by determining sleep profile settings that are more common to those users in a group that have higher sleep quality and less common to those users in the group that have lower sleep quality.

The sleep profiles may also be changed over time based on learning and/or correlation processes, some of which may be performed by or in conjunction with the server. Some of the learning processes may be used to modify sleep profiles to optimize particular aspects of sleep, for example total amount of sleep per sleep session, total amount of sleep-in particular sleep stages, or user reported perception of sleep quality. In some embodiments the server may propagate sleep profile test settings to sleep platforms, with the sleep platforms using the test settings in place of settings otherwise used by the sleep platform. User sleep quality with the test settings may be compared to user sleep quality with the settings otherwise used, so as to determine whether the test settings provide the user with higher sleep quality than the otherwise used settings. In some embodiments the server may make the determinations, in some embodiments the sleep platform may make the determinations, with the sleep platform providing results of the determination to the server. In some embodiments the server may increase propagation to sleep platforms of sleep profile test settings that result in higher quality sleep and decrease propagation of test settings that do not result in higher quality sleep. In some embodiments the sleep profile test settings may be used for varying periods of time depending on the user or user group characteristics, for example in order to obtain a more accurate measurement of the impact of the test settings on sleep quality. In some embodiments the period of time that the test setting is measured can also be part of the sleep profile.

In some embodiments the server may modify sleep profiles to increase specific sleep quality metrics selected by users. In some embodiments, the server may receive target outcomes selected by the users, receive information regarding extent of achievement of the target outcomes by the users, and modify sleep profiles for some users based on sleep profile information of other users based on relative extent of achievement of the target outcomes. For sleep-based target outcomes, the server may determine extent of achievement of the target outcomes based partly or wholly on sleep information provided by the sleep systems.

In some embodiments the server may modify sleep profiles to increase specific sleep quality metrics selected by users based on changing sleep outcomes of the user. For instance, if a user's sleep quality using a given sleep profile starts to worsen over time, the server may determine that different test sleep profiles should be used and evaluated to improve sleep quality. In some embodiments, the server may modify sleep profiles to increase specific sleep quality metrics selected by users based on modified user feedback, needs or requirements. For instance, a user may indicate that they have injured their back or are now pregnant or are experiencing shoulder pain or waking up hot. Given these changing needs or conditions, the server may evaluate sleeper profiles with other users that have been effective in improving sleep quality for similar conditions or needs and use those profiles as test profiles for the user to improve the user's sleep quality. In some embodiments, the server may receive target outcomes selected by the users, receive information regarding extent of achievement of the target outcomes by the users, and modify sleep profiles for some users based on sleep profile information of other users based on relative extent of achievement of the target outcomes. For sleep-based target outcomes, the server may determine extent of achievement of the target outcomes based partly or wholly on sleep information provided by the sleep systems.

The server may receive target outcomes from compute devices associated with users, with the target outcomes selected or entered into the compute devices by the users. The compute devices may be, for example, smartphones, computers, or tablets, and a program or application running on the compute device may provide an appropriate user interface. In some embodiments the compute devices may communicate the target outcomes to the user's sleep platform, for communication to the server, or the compute devices may more directly communicate with the server.

The server may also receive information regarding user sleep status from the sleep platforms. In some embodiments the user sleep status information may be information of sensors of the sleep platform or other sensors associated with the user, and the server may determine user sleep states and sleep events based on such information. In some embodiments the sleep platform may determine user sleep states and sleep events based on information from the sensors, with the sleep platform providing the determined information to the server. In some embodiments the server may receive some sensor information and some information determined by the sleep platform, with the server determining further user sleep state and sleep events.

Extent of achievement for some target outcomes may be determinable by the server based on the information regarding user sleep status provided by the sleep platforms. For example, if a target outcome is to minimize a number of wake events at night, the server may determine a number of wake events of a user based on the user sleep status information (for example using pressure sensor data indicating motion). In such cases, the server may determine the extent of achievement of target outcomes without conscious user input.

In some embodiments the server determines, on a target outcome basis, users who meet a predefined criteria relating to extent of achievement. In some embodiments the predefined criteria is progress based, in that the predefined criteria is greatest progress towards the target outcome. In some embodiments the predefined criteria is achievement based, in that the predefined criteria is greatest extent of achievement. In some such embodiments, the determined users are those users who are above a predetermined percentile. In some such embodiments the determined users may be different for each particular user, with the determined users being all those who have achieved greater progress towards the target outcome or who have achieved a greater extent of achievement towards the target outcome.

For the determined users, the server may determine statistical correlations between settings of the sleep profiles for those users and modify sleep profiles of other users based on the correlated settings.

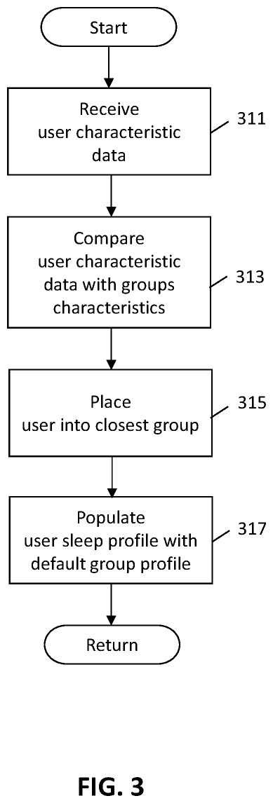

FIG. 3 is a flow chart of a process for determining settings for conditioning an environment of a sleep platform based on user characteristics, in accordance with aspects of the invention. In some embodiments the process is performed by at least one server, for example the server of FIG. 1 or FIG. 2. In some embodiments the process is performed by a system, for example the system of FIG. 1 or FIG. 2. In some embodiments the process is performed by at least one processor, for example as programmed by program instructions.

In block 311 the process receives user characteristic information and/or sleep platform capability information. In some embodiments the sleep platform capability information comprises sleep platform model identification information, which the process may correlate to sleep platform capability information. In some embodiments the user characteristic information includes information such as a user's gender, age, weight, and, in some embodiments, other characteristics such as resting pulse rate, height, and blood pressure. In some embodiments the user characteristic information may include information such as whether the user sleeps alone in the bed or with a companion, sleep preferences as to ambient temperature, sleep surface firmness, type of blanket usage, and other factors. In some embodiments the characteristic information may also include information relating to the user's current health or existing physical conditions, for example temporary conditions such as pregnancy or chronic conditions such as diabetes. In some embodiments the user characteristic information may include information related to a circadian rhythm type of the user. In some embodiments the user characteristic information may include information related to a sleep chronotype of the user. In some embodiments the user characteristic information may include information relating to a user's geographic location. In some embodiments the user characteristic information may include information relating to the user's typical sleep schedule. In some embodiments the user characteristic information may include historical sleep information that the user has recorded and is accessible to the sleep platform system to access, analyze and/or evaluate. In some embodiments the user characteristic information may include health tracker information that the user has recorded and is accessible to the sleep platform system to access, analyze and/or evaluate.

In some embodiments the server provides a fillable form to a computer, tablet, or smartphone of the user, with the user to enter requested information into the computer, tablet, or smartphone. Upon completion, or in response to an input to the computer, tablet, or smartphone, the computer tablet, or smartphone may transmit the entered information to the server.

In block 313 the process compares the user characteristic information and/or sleep platform capability information with group characteristic information. In some embodiments the server compares the user characteristic information with group characteristic information. In some embodiments a plurality of groups are defined, with group definitions being based on examples of user characteristic information. For example, a first group may be defined as including men over a certain age, with particular physical and sleep characteristics, while a second group may be defined as women over a certain age, with a weight/height ratio below a predetermined value. In some embodiments the process compares the user characteristic information with information of group definitions and assigns the user to the group which has a group definition most closely related to the user characteristic information. Correlations between the user and various group definitions may be measured by various degrees of correlation, and the process can include different threshold criteria based on various correlation factors. In some embodiments the group definition may include less information than provided by the user characteristic information. In some embodiments greater weight may be given to some of the information than other of the information in determining the group definition most closely related to the user characteristic information.

In block 315 the process assigns the user into the group with characteristics closest to that of the user. In some embodiments the server assigns the user into the group with the characteristics closest to that of the user. In some embodiments, the server assigns the user to multiple groups that have good correlation with various characteristics, and the server may evaluate and generate multiple sleep profiles from the multiple groups that could be used and tested with the user to improve their sleep quality.

In block 317 the process generates a sleep profile for the user based on a default sleep profile for the group to which the user has been assigned. In some embodiments the default sleep profile is an optimized sleep profile for the group. In some embodiments the server generates the sleep profile for the user. In some embodiments the sleep profile is the default sleep profile for the group.

The process thereafter returns.

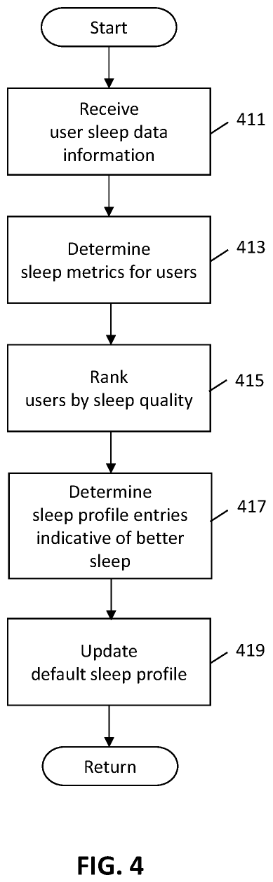

FIG. 4 is a flow chart of a process for updating a sleep profile for a user based on sleep profile information of other users with high sleep quality, in accordance with aspects of the invention. In some embodiments the process is performed by at least one server, for example the server of FIG. 1 or FIG. 2. In some embodiments the process is performed by a system, for example the system of FIG. 1 or FIG. 2. In some embodiments the process is performed by at least one processor, for example as programmed by program instructions. In some embodiments the process determines, or modifies, a default sleep profile for a group.

In block 411 the process receives sleep data for a plurality of users. In some embodiments controllers of the sleep platforms receive the sleep data, and/or generate the sleep data, or some of it, based on information from sensors of the sleep platform. In some embodiments the sleep data is, in addition or instead, provided by sensors worn by the users or otherwise independent of the sleep platforms, but associated with the users. In some embodiments the sleep data is, in addition or instead, entered into a computers, tablets, or smartphones by users, with the sleep data for example being user perceptions of quality and/or quantity of sleep. In some embodiments a server receives the sleep data. In some embodiments the server receives the sleep data from sleep platforms and/or sensors worn by or associated with users and/or computers, tablets, or smartphones associated with the users.

In block 413 the process determines sleep metrics for the users. The sleep metrics are based on the sleep data. In some embodiments the sleep metrics are determined by the server. In some embodiments the sleep metrics indicate quality of sleep. In some embodiments the sleep metrics include one, some, or all of a total sleep time, a time taken to fall asleep, a number of wake occurrences during a sleep session, the length of sleep interruptions, sleep efficiency, an amount of sleep in one or more particular sleep stages, and a perception of sleep quality by the user.

In block 415 the process ranks the users by sleep quality. In some embodiments the server ranks the users by sleep quality. In some embodiments the users are ranked in a sequential order, from highest sleep quality to lowest sleep quality. In some embodiments the users are ranked in tiers, from a highest tier to a lowest tier. In some embodiments each tier includes a predetermined percentage of sleepers. In some embodiments the process separately ranks the users in each group by sleep quality.

In block 417 the process determines sleep profile entries indicative of higher quality sleep. In some embodiments the server determines sleep profile entries indicative of higher quality sleep. In some embodiments sleep profile entries that are common to those with higher quality sleep and less common to those with lower quality sleep are the sleep profile entries indicative of higher quality sleep. In some embodiments regression testing is performed to determine the sleep profile entries indicative of higher quality sleep. In some embodiments the sleep profile entries indicative of higher quality sleep are determined for each group of users.

In block 419 the process updates a default sleep profile. In some embodiments the server updates the default sleep profile. In some embodiments the default sleep profile is updated to reflect the sleep profile entries indicative of higher quality sleep. In some embodiments the default sleep profiles for the different groups are separately updated, for example using sleep profile entries indicative of higher quality sleep for each group. In some embodiments the sleep profile entries indicative of higher quality sleep are also used to update sleep profiles of users generally, in some embodiments on a group-by-group basis. In some embodiments the sleep profile entries indicative of higher quality sleep used in testing whether the entries provide for higher sleep quality for users. In such embodiments, the sleep profile entries indicative of higher quality sleep may be provided to sleep platforms for use, with the sleep platforms, or the server, determining whether those entries provide for higher quality sleep.

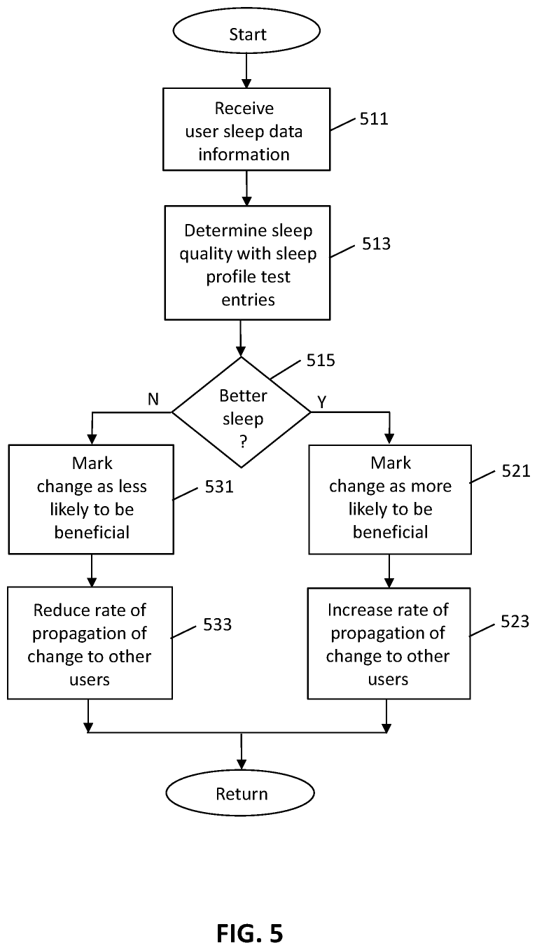

FIG. 5 is a flow diagram of a process for determining test sleep profile parameters based on sleep quality results from a plurality of users, in accordance with aspects of the invention. In some embodiments the process is performed by at least one server, for example the server of FIG. 1 or FIG. 2. In some embodiments the process is performed by a system, for example the system of FIG. 1 or FIG. 2. In some embodiments the process is performed by at least one processor, for example as programmed by program instructions.

In block 511 the process receives sleep data for a plurality of users who have had sleep profile test entries utilized. In some embodiments the server receives the sleep data. In some embodiments controllers of the sleep platforms receive sleep data and/or generate the sleep data, or some of it, based on information from sensors of the sleep platform, and provide the sleep data to the server. In some embodiments the sleep data is, in addition or instead, provided by sensors worn by the users or otherwise independent of the sleep platforms, but associated with the users. In some embodiments the sleep data is, in addition or instead, entered into a computers, tablets, or smartphones by users, with the sleep data for example being user perceptions of quality and/or quantity of sleep.

In block 513 the process determines user sleep quality with use of the sleep profile test entries. In some embodiments the server determines user sleep quality with use of the sleep profile test entries. In some embodiments the server determines sleep quality based on one or more of an amount of sleep, an amount of sleep in a particular sleep stage, a period of time taken to fall asleep, sleep efficiency, the number and length of sleep interruptions, and/or a user perception of sleep quality.

In block 515 the process determines whether use of the test entries resulted in higher quality sleep over the test period. If use of the test entries resulted in higher quality sleep the process continues to block 521. Otherwise, the process continues to block 531.

In block 521 the process marks the test entries as more likely to be beneficial. In block 523 the process increases rate of propagation of the test entries to users. In some embodiments the server increases a number of sleep platforms which are to receive the test entries for testing in a predetermined period of time. In some embodiments the process may modify a default sleep profile to use the test entries in place of entries that would otherwise be used. In some embodiments the process may modify a default sleep profile to use the test entries in place of entries that would otherwise be used in response to the rate of propagation exceeding a first predetermined rate.

In block 531 the process marks the test entries as less likely to be beneficial. In block 533 the process decreases rate of propagation of the test entries to users. In some embodiments the process decreases a number of sleep platforms which are to receive the test entries for testing in a predetermined period of time. In some embodiments the process may cease propagation of the test entries in response to the rate of propagation dropping below a second predetermined rate

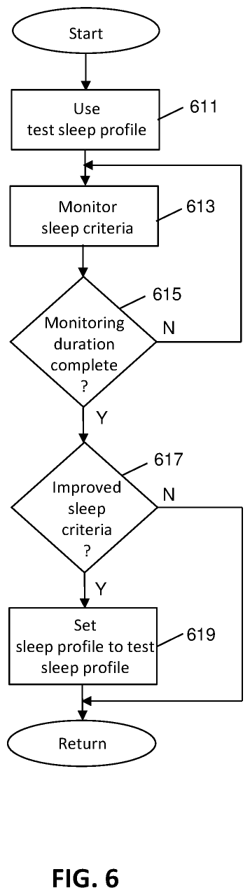

FIG. 6 is a flow diagram of a process for testing sleep profile changes for a user, in accordance with aspects of the invention. In some embodiments the process provides for adjusting parameters for use in conditioning an environment of a sleep platform. In some embodiments the process is performed by a system, for example the system of FIG. 1 or FIG. 2.

In some embodiments the process is performed by a sleep platform and a server, for example a sleep platform and server of FIG. 1 or FIG. 2. In some embodiments the process is performed by a sleep platform, for example the sleep platform of FIG. 1 or one of the sleep platforms of FIG. 2. In some embodiments the process is performed by a bed with adjustable components. In some embodiments the process is performed by a controller of a bed with a pressure adjustment component. In some embodiments the process is performed by a controller of a bed with a temperature adjustment component. In some embodiments the process is performed by a controller of a bed that controls other room environment factors such as lighting, room temperature, room humidity, audio, or video media content accessible in the room. In some embodiments the process is performed by a controller, which may be a processor, and in other embodiments, this computation is performed on a remote server coupled to the controller over a network. In some embodiments the processor receives information indicative of pressure on a sleep surface of a bed. In some embodiments the processor receives information from pressure sensors indicating pressure on at least a portion of the sleep surface, and in some embodiments the processor receives information from biometric sensors of the bed or associated with a user of the bed.

In block 611 the process commands adjustment of the sleep environment of the sleep platform in accordance with test entries in a sleep profile. In some embodiments the test entries may relate to one, some or all firmness of a sleep surface, temperature of the sleep surface, generation of aroma about the sleep platform, or other conditions. In some embodiments the process uses the test entries in place of other entries in a sleep profile for the user.

In block 613 the process monitors or measures one or more sleep criterion or metric. For example, the process may monitor a time taken to fall asleep once a sleeper enters the bed. Also, for example, the process may monitor a time a total time a sleeper has slept during the night. Also, for example, the process may monitor a total time of deep wave sleep the sleeper had during the night. In addition, other sleep related criteria or metrics may be used. In some embodiments the process measures the sleep criteria or metrics based on information from sensors. In some embodiments the process measures the sleep criteria or metrics based on information from biometric sensors.

In block 615 the process determines if the monitoring duration time is complete. In some embodiments the monitoring duration encompasses an entire night's sleep. In some embodiments the monitoring duration encompasses a part of a night's sleep. In some embodiments the monitoring duration is complete once the sleeper falls asleep. In some embodiments the monitoring duration extends to multiple sleeper nights. If the monitoring duration is complete the process continues to block 617, otherwise the process continues measuring sleep criteria in block 613.

In block 617 the process determines if the measured criteria or metrics indicates an improvement in sleep for the sleeper compared to previously measured criteria or metrics for the sleeper using the pressure profile that would otherwise have been used. In some embodiments an improvement in sleep performance is falling asleep faster. In some embodiments an improvement in sleep performance is a longer duration asleep during the night. In some embodiments an improvement in sleep performance is reduced interruption to sleep during the night. In some embodiments an improvement in the user's subjective perceived sleep improvement based on their responses to evaluation questions on restorative sleep quality. In some embodiments an improvement in the user's subjective perceived sleep improvement based on their responses to predetermined evaluation questions on restorative sleep quality In some embodiments the process may repeat the operations of block 611-615 multiple times, for example over multiple nights, and use an average, or some other statistically calculated value, for determining whether the sleeper experienced an improvement in sleep with the test entries used rather the sleep profile entries that would have been otherwise used.

If an improvement in sleep performance occurred, the process continues to block 619. Otherwise, the process returns.

In block 619 the process replaces the sleep profile entries that would have been otherwise used with the test entries. In some embodiments the process provides a notification to a server that use of the test entries provide higher quality sleep than the sleep profile entries that would have been otherwise used.

The process thereafter returns.

Although the invention has been discussed with respect to various embodiments, it should be recognized that the invention comprises the novel and non-obvious claims supported by this disclosure.

What is claimed is:

1. A method of setting an aspect of a sleep environment of a sleep platform for a user, comprising:
determining the sleep quality of the user for a given sleep environment setting;
comparing the user's sleep quality with the sleep quality of users in a sleep user group of sleep users having at least one similar characteristic;
modifying a setting of the sleep environment of the sleep platform for the user based on settings of sleep environments of users in the sleep user group indicated as providing improved sleep quality for at least some other users in the sleep user group.

2. The method of claim 1, wherein the aspect of the sleep environment of the sleep platform for the user is set based on sleep environment information for those of the other users in the sleep user group whose sleep quality exceeds that of the user.

3. The method of claim 2 wherein sleep quality is determined by one or more measurements that may be made of the user and the other users during sleep sessions of those users.

4. The method of claim 2 wherein sleep quality is determined by responses by users to one or more questions that may be presented to the user and the other users after sleep sessions of those users.

5. The method of claim 1, wherein at least one server receives information regarding status of the sleep platforms, the at least one server receives metrics indicating quality of sleep for the plurality of users of sleep platforms, and the at least one server determines modifications to the status of at least one of the sleep platforms by selecting settings for the at least one of the sleep platforms based on settings for sleep platforms of users with a sleep quality of at least a predetermined level.

6. A method for modifying sleep platform settings, comprising:
collecting sleep related data for a plurality of users, the plurality of users having at least one similar characteristic;
determining sleep profile settings indicative of higher sleep quality based on at least some of the sleep related data, and
modifying a sleep environment for a sleep platform using the sleep profile settings indicative of higher sleep quality.

7. The method of claim 6, wherein sleep profile settings indicative of higher sleep quality are propagated to one or more sleep platforms for use in testing whether those profile settings result in higher quality sleep for users of the one or more sleep platforms.

8. A method of setting an aspect of a sleep environment of a sleep platform for a user, comprising:
determining the sleep quality of the user for a given sleep environment setting;
comparing the user's sleep quality with the sleep quality of users in a sleep user group of sleep users using a similar sleep platform;
modifying the setting of the sleep environment of the sleep platform for the user based on settings of sleep environments of users in the sleep user group indicated as providing improved sleep quality for at least some other users in the sleep user group.

9. The method of claim 8, wherein the aspect of the sleep environment of the sleep platform for the user is set based on sleep environment information for those of the other users in the sleep user group whose sleep quality exceeds that of the user.

10. The method of claim 8, wherein the users in the sleep user group have at least one similar characteristic as the user.

11. The method of claim 9 wherein sleep quality is determined by one or more measurements that may be made of the user and the other users during sleep sessions of those users.

12. The method of claim 9 wherein sleep quality is determined by responses by users to one or more questions that may be presented to the user and the other users after sleep sessions of those users.

13. The method of claim 8, wherein at least one server receives information regarding status of the sleep platforms, the at least one server receives metrics indicating quality of sleep for the plurality of users of sleep platforms, and the at least one server determines modifications to the status of at least one of the sleep platforms by selecting settings for the at least one of the sleep platforms based on settings for sleep platforms of users with a sleep quality of at least a predetermined level.
